(19) United States
(12) Patent Application Publication
(10) Pub. No.: US 2009/0005348 A1
(43) Pub. Date: Jan. 1, 2009

(54) Compounds Having Cytokine Modulating Properties

(75) Inventors: Esra Ogru, Victoria (AU); Roksan Libinaki, Victoria (AU); Robert Gianello, Victoria (AU)

Correspondence Address: MICHAEL BEST & FRIEDRICH LLP, 100 E WISCONSIN AVENUE, Suite 3300, MILWAUKEE, WI, 53202, US

(73) Assignee: VITAL HEALTH SCIENCES PTY LTD, Melbourne, Victoria (AU)

(21) Appl. No.: 12/158,932

(22) PCT Filed: Dec. 22, 2006
(86) PCT No.: PCT/AU06/01997

§ 371 Date: Aug. 15, 2008

(30) Foreign Application Priority Data
Dec. 23, 2005 (AU) 2005907306
Mar. 8, 2006 (AU) 2006901179

(51) Int. Cl. A61K 31/665; A61P 37/02

(52) U.S. Cl. 514/100

(57) ABSTRACT

A method of modulating one or more immuno-regulatory cytokines, such as pro-inflammatory and/or anti-inflammatory cytokines, comprising administering to a subject a therapeutically effective amount of one or more phosphate derivatives of one or more hydroxy chromans, or complexes thereof.

### TECHNICAL FIELD

The present invention relates to compounds which have cytokine modulating properties.

### RELATED APPLICATIONS

The present application claims priority from Australian Provisional Patent Application No. 2005907306 filed on 23 Dec. 2005 and Australian Provisional Patent Application No. 2006901179 filed on 8 Mar. 2006, the entire disclosures of which are hereby incorporated by reference.

### BACKGROUND

In this specification, where a document, act or item of knowledge is referred to or discussed, this reference or discussion is not an admission that the document, act or item of knowledge or any combination thereof was at the priority date: part of common general knowledge, or known to be relevant to an attempt to solve any problem with which this specification is concerned.

Cytokines are a large group of molecules that regulate interactions in the immune system. Cytokines are messengers that carry biochemical signals to regulate local and systemic immune responses, inflammatory reactions, wound healing, formation of blood cells, and many other biological processes. More than 100 cytokines have been identified.

Cytokines must be produced de novo in response to an immune stimulus. They generally (although not always) act over short distances and short time spans and at very low concentration. They act by binding to specific membrane receptors, which then signal the cell via second messengers, often tyrosine kinases, to alter its behaviour (gene expression). Responses to cytokines include increasing or decreasing expression of membrane proteins (including cytokine receptors), proliferation, and secretion of effector molecules.

Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action).

Chemokines are a family of small cytokines, or proteins secreted by cells. Chemokines induce directed chemotaxis in nearby responsive cells. Some chemokines are considered pro-inflammatory and can be induced during an immune response while others are considered homeostatic. Inflammatory chemokines are released from a wide variety of cells in response to bacterial infection, viruses and agents that cause physical damage. They function mainly as chemoattractants for leukocytes, recruiting monocytes, neutrophils and other effector cells from the blood to sites of infection or damage. They can be released by many different cell types and serve to guide cells involved in innate immunity and also the lymphocytes of the adaptive immune system. Some chemokines also have roles in the development of lymphocytes, migration and angiogenesis (the growth of new blood vessels).

Lymphokines are a subset of cytokines that are produced by immune cells.

Monokines are soluble cytokines that mediate immune responses. Monokines are the products of mononuclear phagocytes and have regulatory effects on lymphocyte function.

It is common for different cell types to secrete the same cytokine or for a single cytokine to act on several different cell types (pleitropy). Cytokines are redundant in their activity, meaning similar functions can be stimulated by different cytokines. Cytokines are often produced in a cascade, as one cytokine stimulates its target cells to make additional cytokines. Cytokines can also act synergistically (two or more cytokines acting together) or antagonistically (cytokines causing opposing activities).

Cytokine activities are characterized using recombinant cytokines and purified cell populations in vitro, or with knock-out mice for individual cytokine genes to characterize cytokine functions in vivo. Cytokines are made by many cell populations, but the predominant producers are helper T cells (Th) and macrophages.

Pro-inflammatory cytokines generally stimulate inflammatory responses, which in turn cause many of the clinical problems associated with immune deficiency and autoimmune disorders. Cytokines are therefore critical to the functioning of both innate and adaptive immune responses. Apart from their importance in the development and functioning of the immune system, cytokines play a major role in a variety of immunological, inflammatory and infectious diseases. As a result of these activities, cytokines are also thought to play a role in cell proliferative disorders including cancer and tumour growth.

The participation of cytokines in immune responses and inflammation also has implications for a role of these proteins in cancer. A causal relationship between inflammation and cancer has long been suspected. Indeed the presence of leukocytes in malignant tissue has been demonstrated, and it has therefore been claimed that some tumours arise from regions of chronic inflammation.

The microenvironment in and around tumours contains cells of the innate immune system. This environment enhances cell proliferation, migration and survival, as well as enhancing angiogenesis, which ultimately promotes tumour development. Furthermore, the inflammatory response is similar in many respects to a wound-healing response, and tumours have been considered as wounds that do not heal.

Chronic infection and consecutive inflammation may directly affect the cells that eventually become transformed. For example in MALT lymphoma, chronic infection may cause persistent B cell activation culminating in chromosomal rearrangements which cause cancer. There also exists an indirect mechanism, for example in epithelial-derived tumours, where inflammation stimulates tumour growth through an indirect mechanism involving activation of surrounding inflammatory cells.

Therefore, the ability to modulate the function of cytokines provides a mechanism for the treatment of disorders which arise from aberrant cytokine activity.

### SUMMARY

It has now been found that phosphate derivatives of hydroxy chromans, or complexes thereof, modulate the function of cytokines, in particular immuno-regulatory cytokines, and thus are useful in the treatment and/or prophylaxis of disorders which arise from aberrant cytokine activity.

In a first aspect, there is provided a method of modulating one or more immuno-regulatory cytokines comprising administering to a subject a therapeutically effective amount of one or more phosphate derivatives of one or more hydroxy chromans, or complexes thereof.

There is also provided use of one or more phosphate derivatives of one or more hydroxy chromans, or complexes thereof, for modulating one or more immuno-regulatory cytokines.

There is further provided use of one or more phosphate derivatives of one or more hydroxy chromans, or complexes thereof, in the manufacture of a medicament to modulate one or more immuno-regulatory cytokines.

In a preferred embodiment, the immuno-regulating cytokines are pro-inflammatory cytokines and/or anti-inflammatory cytokines.

Immuno-regulatory cytokines modulate interactions in the immune system, e.g. regulate immune function and processes by inhibiting an inflammatory response and/or stimulating an anti-inflammatory response.

In a second aspect of the invention, there is provided a method of inhibiting an inflammatory response and/or stimulating an anti-inflammatory response comprising administering to a subject a therapeutically effective amount of one or more phosphate derivatives of one or more hydroxy chromans, or complexes thereof.

There is also provided use of one or more phosphate derivatives of one or more hydroxy chromans, or complexes thereof, for inhibiting an inflammatory response and/or stimulating an anti-inflammatory response.

There is further provided use of one or more phosphate derivatives of one or more hydroxy chromans, or complexes thereof, in the manufacture of a medicament to inhibit an inflammatory response and/or stimulating an anti-inflammatory response.

Particular disorders which arise from aberrant cytokine activity include immune disorders, inflammatory disorders, and cellular proliferative disorders.

In a third aspect, there is provided a method of treatment and/or prophylaxis of immune disorders, inflammatory disorders, and/or cellular proliferative disorders, comprising administering to a subject a therapeutically effective amount of one or more phosphate derivatives of one or more hydroxy chromans, or complexes thereof.

There is also provided use of one or more phosphate derivatives of one or more hydroxy chromans, or complexes thereof, for the treatment and/or prophylaxis of immune disorders, inflammatory disorders, and/or cellular proliferative disorders.

There is further provided use of one or more phosphate derivatives of one or more hydroxy chromans, or complexes thereof, in the manufacture of a medicament for the treatment and/or prophylaxis of immune disorders, inflammatory disorders, and/or cellular proliferative disorders.

In a fourth aspect, there is provided an immune-modulator agent, anti-inflammatory agent, or anti-cancer agent, comprising one or more phosphate derivatives of one or more hydroxy chromans, or complexes thereof.

There is also provided use of one or more phosphate derivatives of one or more hydroxy chromans, or complexes thereof, as an immune-modulator agent, anti-inflammatory agent, or anti-cancer agent.

There is further provided one or more phosphate derivatives of one or more hydroxy chromans, or complexes thereof, for use as an immune-modulator agent, anti-inflammatory agent, or anti-cancer agent.

It will be appreciated that the term “immune-modulator agent” encompasses “immune-stimulator agents” as well as “immune-suppressant agents”.

It will also be appreciated that the term “anti-cancer” includes “anti-tumour”.

### DETAILED DESCRIPTION

The present invention relates to phosphate derivatives of hydroxy chromans, or complexes thereof, which modulate the function of cytokines, in particular immuno-regulatory cytokines, and thus provide a mechanism in the treatment and/or prophylaxis of disorders which arise from aberrant cytokine activity.

### Hydroxy Chromans

The term “hydroxy chromans” is used herein to refer to the hydroxy derivatives of chromans.

The hydroxy chroman derivatives include all isomers of the tocols and tocotrienols, whether in enantiomeric or racemic forms.

The tocols include all isomers of derivatives of 6:hydroxy 2:methyl chroman having the formula (I) below including α-5:7:8 tri-methyl, β-5:8 di-methyl, γ-7:8 di-methyl, and δ-8 methyl derivatives.

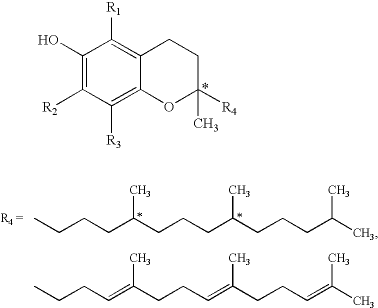

in which

R₁, R₂ and R₃ are independently selected from the group consisting of hydrogen and C₁₋₆ alkyl, preferably methyl.

The term “C₁₋₆ alkyl” when used either alone or in combination (e.g. C(═O)—C₁₋₆ alkyl) refers to straight chain or branched chain hydrocarbon groups having from 1 to 6 carbon atoms. Examples include ethyl, propyl, isopropyl, butyl, isobutyl, sec-butyl, tert-butyl, pentyl, neopentyl, and hexyl.

In the tocopherols, R₄ is substituted by 4:8:12 tri-methyl tridecane (see above) and the 2, 4, and 8 positions (see *) may be stereoisomers with R or S activity or racemic.

In the tocotrienols, R₄ is substituted by 4:8:12 tri-methyl trideca-3:7:11 triene (see above) and the 2 position (see *) may be stereoactive as R or S stereoisomers or racemic.

In a preferred embodiment, the hydroxy chroman derivative is selected from the group consisting of α, β, δ, and γ tocols, and mixtures thereof, more preferably, α-tocopherol or tocotrienol.

### Phosphate Derivatives of Hydroxy Chromans

The term “phosphate derivatives” is used herein to refer to the acid forms of phosphorylated hydroxy chromans, salts of the phosphates including metal salts such as sodium, magnesium, potassium and calcium, and any other derivative where the phosphate proton is replaced by other substituents such as, for example, C₁₋₆ alkyl or phosphatidyl groups.

In some situations, it may be necessary to use a phosphate derivative such as a phosphatide where additional properties, such as increased water solubility, are preferred. Phosphatidyl derivatives are amino alkyl derivatives of organic phosphates. These derivatives may be prepared from amines having a structure of R₁R₂N(CH₂)ₙOH wherein n is an integer between 1 and 6 and R₁ and R₂ may be either H or C₁₋₆ alkyl. R₁ and R₂ may be the same or different. The phosphatidyl derivatives are prepared by displacing the hydroxyl proton of the hydroxy chromans with a phosphate entity that is then reacted with an amine, such as ethanolamine or N,N′ dimethylethanolamine, to generate the phosphatidyl derivative of the hydroxy chroman. One method of preparation of the phosphatidyl derivatives uses a basic solvent such as pyridine or triethylamine with phosphorous oxychloride to prepare the intermediate which is then reacted with the hydroxy group of the amine to produce the corresponding phosphatidyl derivative, such as P cholyl P tocopheryl dihydrogen phosphate.

The phosphate derivatives of hydroxy chromans are selected from the group consisting of mono-tocopheryl phosphate derivatives, di-tocopheryl phosphate derivatives, mono-tocotrienyl phosphate derivatives, di-tocotrienyl phosphate derivatives, and mixtures thereof. In preferred embodiments, the phosphate derivatives of hydroxy chromans are a mixture of mono-tocopheryl phosphate derivatives, di-tocopheryl phosphate derivatives, mono-tocotrienyl phosphate derivatives, and/or di-tocotrienyl phosphate derivatives. In one preferred embodiment, the phosphate derivatives of hydroxy chromans are a mixture of mono-tocopheryl phosphate derivatives and di-tocopheryl phosphate derivatives, most preferably a mixture of mono-tocopheryl phosphate (TP) and di-tocopheryl phosphate (T2P).

The ratio of mono-tocopheryl phosphate (TP) to di-tocopheryl phosphate (T2P) is preferably 4:1 to 1:4, more preferably 2:1 to 1:2, most preferably 2:1.

### Complexes of Phosphate Derivatives of Hydroxy Chromans

In some situations, complexes of phosphate derivatives of hydroxy chromans may also be utilized where additional properties such as improved stability or deliverability may be useful.

The term “complexes of phosphate derivatives of hydroxy chromans” refers to the reaction product of the phosphate derivatives of hydroxy chromans with one or more complexing agents selected from the group consisting of amphoteric surfactants, cationic surfactants, amino acids having nitrogen functional groups and proteins rich in these amino acids. Examples of proteins rich in amino acids having nitrogen functional groups are proteins having either at least 1 in 62 amino acids as arginine, or at least 1 in 83 histidine, or at least 1 in 65 as lysine, such as the various forms of the protein casein.

Preferred complexing agents are selected from the group consisting of amino acids such as arginine and lysine, and tertiary substituted amines, such as those of formula (II):

NR⁷R⁸R⁹ (II)

in which

R⁷ is selected from the group consisting of C₁₋₂₂ alkyl optionally interrupted by carbonyl; and

R⁸ and R⁹ are independently selected from the group consisting of H, CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X in which X is H, Na, K or alkanolamine,

provided R⁸ and R⁹ are not both H and when R⁷ is RCO, then R⁸ is CH₃ and R⁹ is (CH₂CH₂)N(C₂H₄OH)—H₂CHOPO₃ or R⁸ and R⁹ together is N(CH₂)₂N(C₂H₄OH)CH₂COO—.

The term “C₁₋₂₂ alkyl” refers to straight chain or branched chain hydrocarbon groups having 1 to 22 carbon atoms, or cyclic hydrocarbon groups having from 6 to 22 carbon atoms. Examples include hexyl, cyclohexyl, decyl, dodecyl, tridecyl, tetradecyl, pentadecyl, hexadecyl, heptadecyl, and octadecyl.

Preferred complexing agents include arginine, lysine and/or lauryliminodipropionic acid where complexation occurs between the alkaline nitrogen centre and the phosphoric acid ester to form a stable complex.

### Complexing Agents Suitable for Combination Therapy

Particular proteins can be used as complexing agents when combination therapy is desired. Examples of such proteins include insulin, parathyroid hormone (PTH), glucagon, calcitonin, adrenocorticotropic hormone (ACTH), prolactin, Interferon-α and -β and -γ, leutenising hormone (LH) (also known as gonadotropin releasing hormone), follicle stimulating hormone (FSH), colony stimulating factor (CSF), and growth hormone (GH). The amino acid composition of these examples is listed in the table below.

| | | Ratio of Total |
|---|---|---|
| Amino acids in protein | Amino acids | Amino Acids |
| Insulin | 110 | |
| arg | 5 | 1 in 22 |
| his | 2 | 1 in 55 |
| lys | 2 | 1 in 55 |
| PTH | 84 | |
| arg | 5 | 1 in 17 |
| his | 0 | 0 |
| lys | 5 | 1 in 17 |
| Glucagon | 180 | |
| arg | 16 | 1 in 11 |
| his | 4 | 1 in 45 |
| lys | 10 | 1 in 18 |
| Calcitonin | 93 | |
| arg | 6 | 1 in 16 |
| his | 3 | 1 in 31 |
| lys | 5 | 1 in 19 |
| ACTH | 41 | |
| arg | 3 | 1 in 14 |
| his | 1 | 1 in 41 |
| lys | 4 | 1 in 10 |
| Prolactin | 220 | |
| arg | 12 | 1 in 18 |
| his | 9 | 1 in 13 |
| lys | 11 | 1 in 11 |
| Interferon-alpha and beta | 133 | |
| arg | 7 | 1 in 19 |
| his | 2 | 1 in 83 |
| lys | 7 | 1 in 19 |
| Interferon-gamma | 166 | |
| arg | 8 | 1 in 21 |
| his | 2 | 1 in 83 |
| lys | 21 | 1 in 8 |
| LH | 92 | |
| arg | 5 | 1 in 18 |
| his | 2 | 1 in 46 |
| lys | 7 | 1 in 13 |
| FSH | 129 | |
| arg | 5 | 1 in 26 |
| his | 2 | 1 in 65 |
| lys | 9 | 1 in 14 |
| CSF | 144 | |
| arg | 6 | 1 in 24 |
| his | 3 | 1 in 48 |
| lys | 6 | 1 in 24 |
| GH domain AOD9604 | 16 | |
| arg | 2 | 1 in 8 |

It should also be appreciated that these proteins may also be used as complexing agents when combination therapy is not desired.

It will be appreciated that the term “phosphate derivatives of hydroxy chromans” is sometimes used herein to more generally refer “one or more phosphate derivatives of one or more hydroxy chromans, or complexes thereof”.

### Cytokine Modulation and Activity

Phosphate derivatives of hydroxy chromans act as therapeutic agents to modulate cytokines, in particular immuno-regulatory cytokines, such as for example, pro-inflammatory and anti-inflammatory cytokines. Accordingly, there is provided a method of modulating one or more immuno-regulatory cytokines comprising administering to a subject a therapeutically effective amount of one or more phosphate derivatives of one or more hydroxy chromans, or complexes thereof.

The terms “modulate”, “modulating”, and “modulation” are used herein to refer to a change in a measurable parameter. The parameter is a characteristic that is objectively measured and evaluated as an indicator of normal biological processes, pathogenic processes, or pharmacological responses to therapeutic interventions. For example, in one embodiment, “modulation” may refer to an increase or decrease in the activity of a cytokine compared to the activity of the cytokine prior to modulation. The activity may be increased or decreased by direct binding of a phosphate derivative of a hydroxy chroman to the cytokine, or the activity of the cytokine may be modulated by an indirect mechanism. For example, a phosphate derivative of a hydroxy chroman may lead to an increase or decrease in the expression or activity of proteins with which the cytokine interacts, such as cytokine receptors. In another embodiment, “modulation” may refer to an increase in T cell proliferation compared to the level of proliferation of the T cell prior to modulation.

The term “immuno-regulatory cytokine” refers to cytokines that modulate interactions in the immune system, e.g. regulate immune function and processes by inhibiting an inflammatory response and/or stimulating an anti-inflammatory response. In a preferred embodiment, the immuno-regulatory cytokines are pro-inflammatory and/or anti-inflammatory cytokines.

“Pro-inflammatory cytokines” are immuno-regulatory cytokines that favour inflammation, and are important mediators of inflammation, immunity, proteolysis, cell recruitment and proliferation. The major pro-inflammatory cytokines that are responsible for early responses include Interleukin-type cytokines such as Interleukin-1α (IL-1α), Interleukin-1β (IL-1β), and Interleukin-6 (IL-6), and Tumour Necrosis Factor-type cytokines such as Tumour Necrosis Factor-alpha (TNFα also known as cachexin and cachectin). Other pro-inflammatory mediators include Interleukin-8 (IL-8), Interleukin-11 (IL-11), and Interleukin-18 (IL-18). These act as endogenous pyrogens (IL-1, IL-6, TNFα) to up regulate the synthesis of secondary mediators and pro-inflammatory cytokines by both macrophages and mesenchymal cells (including fibroblasts, epithelial and endothelial cells) and stimulate the production of acute phase proteins or attract inflammatory cells.

“Anti-inflammatory cytokines” are immuno-regulatory cytokines that counteract various aspects of inflammation, for example cell activation or the production of pro-inflammatory cytokines, and therefore contribute to the control of the magnitude of the inflammatory responses in vivo. These mediators act mainly by the inhibition of the production of pro-inflammatory cytokines or by counteracting many biological effects of pro-inflammatory mediators in different ways. The major anti-inflammatory cytokines are Interleukin-type cytokines such as Interleukin-4 (IL-4), Interleukin-10 (IL-10) and Interleukin-13 (IL-13). Other anti-inflammatory cytokines include: Interferon cytokines such as IFNα, Growth Factor cytokines, in particular Transforming Growth Factor cytokines such as TGFβ and Granulocyte-colony Stimulating Factor cytokines such as G-CSF, as well as soluble receptors for TNF or IL-6.

It should be noted that the common and clear-cut classification of immuno-regulatory cytokines as either pro-inflammatory or anti-inflammatory may be misleading. The net effect of an inflammatory response is determined by the balance between pro-inflammatory and anti-inflammatory cytokines. The type, duration and extent of the cellular activities induced by one particular cytokine can be influenced considerably by the nature of the target cells, the micro-environment of the cell, for example, the growth and activation state of the cells, the type of neighbouring cells, cytokine concentrations, the presence of other cytokines and even on the sequence of several cytokines acting on the same cell.

“Interleukin-type cytokines” may generally be described as cytokines made by one leukocyte and acting on other leukocytes. Interleukin-type cytokines can also be further characterized as chemokines, monokines and lymphokines.

“Tumour Necrosis Factor-type cytokines” are potent pro-inflammatory cytokines which are expressed by activated macrophages and lymphocytes. These cytokines induce diverse cellular responses that can vary from apoptosis, to the expression of genes involved in both early inflammatory and acquired immune responses.

“Interferon cytokines” are natural proteins produced by cells of the immune system of most vertebrates in response to challenges by foreign agents such as viruses, bacteria, parasites and tumour cells.

“Growth Factor cytokines” are a group of biologically active poly-peptides which function as hormone-like regulatory signals, controlling growth and differentiation of responsive cells.

Due to the redundancy and pleiotropy of cytokines, they are often produced in a cascade and may also act synergistically or antagonistically, therefore making it difficult for any one cytokine to have a profound effect in vivo.

An inflammatory response is associated with vasodilation, increased vascular permeability, recruitment of inflammatory cells (especially neutrophils in acute inflammation), the release of inflammatory mediators from these cells (including vasoactive amines, prostanoids and reactive oxygen intermediates) and cytokine release. The macrophage-derived cytokines IL-1 and IL-6 are primarily responsible for the acute phase response by causing a protective change in plasma protein production by hepatocytes.

Some of the more important acute phase proteins include:
1. Protease inhibitors (e.g. α1-antitrypsin, antichymotrypsin);
2. Coagulation proteins (e.g. atherosclerosis, fibrinogen, prothrombin and plasminogen);
3. Complement proteins (e.g. C2, C3, C4 and C5);
4. Transport proteins (e.g. haptoglobin and haemopexin);
5. Miscellaneous proteins that do not fall under these groupings including, for example, C-reactive protein (CRP), fibronectin and serum amyloid A.

CRP is a member of the class of acute phase proteins as its levels rise dramatically during inflammatory processes occurring in the body. It is thought to assist in complement binding to foreign and damaged cells and affect the humoral response to disease. It is also believed to play an important role in innate immunity, as an early defense system against infections.

The most common conditions associated with major elevations of CRP levels include:
1. Hypersensitivity complications of infections (e.g. Rheumatic fever);
2. Inflammatory disease (e.g. Rheumatoid arthritis, Reiter's disease, Crohn's disease);
3. Allograft rejection (e.g. renal transplantation);
4. Malignancy (e.g. lymphoma and sarcoma);
5. Necrosis (e.g. myocardial infarction, tumour embolism and acute phase pancreatitis);
6. Trauma (e.g. burns and fractures).

While an elevation of CRP value is not specific for any condition, it is a very sensitive index of ongoing inflammation and so provides a valuable adjunct to a careful clinical assessment. Once a diagnosis has been established, CRP may be used to monitor a patient's response to therapy. Infections monitored by CRP level include: pyelonephritis, pelvic infections, meningitis and endocarditis.

Perhaps the most extreme example of a cytokine related disorder is a cytokine storm, which is a potentially fatal immune reaction consisting of a positive feedback loop between cytokines and immune cells, with highly elevated levels of various cytokines. The cytokine storm is the systemic expression of a healthy and vigorous immune system resulting in the release of more than 150 inflammatory mediators (cytokines, oxygen free radicals, and coagulation factors). Both pro-inflammatory cytokines (such as TNFα, IL-1, and IL-6) and anti-inflammatory cytokines (such as IL-10 and IL-1) are elevated in the serum of patients experiencing a cytokine storm. Cytokine storms can occur in a number of infectious and non-infectious diseases including graft versus host disease (GVHD), adult respiratory distress syndrome (ARDS), sepsis, influenza, and systemic inflammatory response syndrome (SIRS).

### Pharmaceutical Compositions

The administration of the phosphate derivatives of hydroxy chromans may be any suitable means that results in a concentration of the phosphate derivatives of hydroxy chromans that is effective to yield the desired therapeutic or prophylactic response. The phosphate derivatives of hydroxy chromans may be contained in any appropriate amount in any suitable carrier and is generally present in an amount of 1-95% by weight of the total weight of a pharmaceutical composition. The carrier must be “pharmaceutically acceptable” in the sense of being compatible with other ingredients of the composition and not injurious to the subject.

The pharmaceutically acceptance carrier is preferably an organic solvent such as acetone, benzene, acetonitrile, chloroform, canola oil, DMSO or an alcohol, for example, methanol or ethanol. If the phosphate derivatives of hydroxy chromans show a poor solubility in water, when water is combined with an organic solvent a stable mixture is formed.

The phosphate derivatives of hydroxy chromans may additionally be combined with other medicaments to provide an operative combination. It is intended to include any chemically compatible combination of pharmaceutically-active agents, as long as the combination does not eliminate the activity of the phosphate derivatives of hydroxy chromans. It will be appreciated that the phosphate derivatives of hydroxy chromans and the other medicament may be administered separately, sequentially or simultaneously. Other medicaments may include, for example other anti-inflammatory and/or anti-cancer agents.

The composition may be provided in a dosage form that is suitable for oral, parenteral (including intravenous, intramuscular, subcutaneous and intradermal), enteral, rectal, vaginal, nasal, inhalation, topical, or ocular administration routes. Thus, the composition may be in form of tablets, capsules, pills, powders, granulates, suspensions, emulsions, liquids, gels including hydrogels, pastes, ointments, creams, plasters, drenches, delivery devices, suppositories, enemas, injectables, implants, sprays or aerosols. The compositions may be formulated according to conventional pharmaceutical practice (see, for example, Remington: The Science and Practice of Pharmacy, (19^{th} ed.), A R Gennaro, 1995, Mack Publishing Company, Easton, Pa., and Encyclopaedia of Pharmaceutical Technology, eds., J Swarbrick and J C Boylan, 1988-1999, Marcel Dekker, New York).

Compositions may be formulated to release the phosphate derivatives of hydroxy chromans substantially immediately upon administration or at any predetermined time or time period after administration. The latter types of compositions are generally known as controlled release formulations, which include (i) formulations that create a substantially constant concentration of the active compound (i.e. the phosphate derivatives of hydroxy chromans) within the body over an extended period of time; (ii) formulations that after a predetermined lay time create a substantially constant concentration of the active compound within the body over an extended period of time; (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern); (iv) formulations that localise active compound action by, for example, special placement of a controlled release composition adjacent to or in the diseased tissue or organ; and (v) formulations that target active compound action by using carriers or chemical derivatives to deliver the active compound to a particular target cell type.

Administration of the phosphate derivatives of hydroxy chromans in the form of a controlled release formulation is especially preferred in cases in which the phosphate derivatives of hydroxy chromans have (i) a narrow therapeutic index (i.e. the difference between the plasma concentration leading to harmful side effects or toxic reactions and the plasma concentration leading to a therapeutic effect is small; in general, the therapeutic index, TI, is defined as the ratio of median lethal dose (LD₅₀) to median effective dose (ED₅₀)); (ii) a narrow absorption window in the gastro-intestinal tract; or (iii) a very short biological half-like so that frequent dosing during a day is required in order to sustain the plasma level at a therapeutic level.

Any number of strategies can be applied in order to obtain a controlled release formulation in which the rate of release outweighs the rate of metabolism of the phosphate derivatives of hydroxy chromans in question. In one example, controlled release is obtained by appropriate selection of various formulation parameters and ingredients, including, for example, various types of controlled release compositions and coatings. Thus, the phosphate derivatives of hydroxy chromans are formulated with appropriate excipients into a pharmaceutical composition that, upon administration to the subject, releases the phosphate derivatives of hydroxy chromans in a controlled manner. Examples include single or multiple unit tablet or capsule compositions, oil solutions, suspensions, emulsions, microcapsules, microspheres, nanoparticles, patches, and liposomes.

### Solid Dosage Forms for Oral Use

Formulations for oral use include tablets containing the phosphate derivatives of hydroxy chromans in a mixture with non-toxic pharmaceutically acceptable excipients. These excipients may be, for example, inert diluents or fillers (e.g. sucrose, sorbitol, sugar, mannitol, mirocrystalline cellulose, starches including potato starch, calcium carbonate, sodium chloride, lactose, calcium phosphate, calcium sulphate, sodium phosphate); granulating and disintegrating agents (e.g. cellulose derivatives including microcrystalline cellulose, starches including potato starch, croscarmellose sodium, alginates, alginic acid); binding agents (e.g. sucrose, glucose, sorbitol, acacia, alginic acid, sodium alginate, gelatin, starch, pregelatinized starch, microcrystalline cellulose, magnesium aluminium silicate, carboxymethylcellulose sodium, methylcellulose, hydroxypropyl methylcellulose, ethylcellulose, polyvinylpyrrolidone, polyethylene glycol); and lubricating agents, glidants, and antiadhesives (e.g. magnesium stearate, zinc stearate, stearic acid, silicas, hydrogenated vegetable oils, talc). Other pharmaceutically acceptable excipients can be colourants, flavouring agents, plasticisers, humectants, buffering agents, and the like.

Tablets may be uncoated or they may be coated by known techniques, optionally to delay disintegration and absorption in the gastrointestinal tract and thereby providing a sustained action over a longer period. The coating may be adapted to release the phosphate derivatives of hydroxy chromans in a predetermined pattern (e.g. in order to achieve a controlled release formulation) or it may be adapted not to release the phosphate derivatives of hydroxy chromans until after passage of the stomach (i.e. enteric coating). The coating may be a sugar coating, a film coating (e.g. based on hydroxypropyl methylcellulose, methylcellulose, methyl hydroxyethylcellulose, hydroxypropylcellulose, carboxymethylcellulose, acrylate copolymers, polyethylene glycols, polyvinylpyrrolidone), or an enteric coating (e.g. based on methacrylic acid copolymer, cellulose acetate phthalate, hydroxypropyl methylcellulose phthalate, hydroxypropyl methylcellulose acetate succinate, polyvinyl acetate phthalate, shellac, ethylcellulose). Furthermore, a time delay material such as, glyceryl monostearate, or glyceryl distearate, may be employed.

Solid tablet compositions may include a coating adapted to protect the composition from unwanted chemical changes (e.g. chemical degradation prior to the release of the phosphate derivatives of hydroxy chromans). The coating may be applied on the solid dosage form in a similar manner as that described in Encyclopaedia of Pharmaceutical Technology, supra.

Formulations for oral use may also be presented as chewing tablets or as hard gelatin capsules wherein the phosphate derivatives of hydroxy chromans are mixed with an inert solid diluent (e.g. potato starch, lactose, microcrystalline cellulose, calcium carbonate, calcium phosphate, kaolin), or as soft gelatin capsules wherein the phosphate derivatives of hydroxy chromans are mixed with water or an oil medium, for example, peanut oil, liquid paraffin or olive oil. Powders and granulates may be prepared using the ingredients mentioned above under tablets and capsules in a conventional manner using, for example, a mixer, a fluid bed apparatus or a spray drying equipment.

### Liquids for Oral Administration

Powders, dispersible powders, or granules suitable for preparation of an aqueous suspension by addition of water are convenient dosage forms for oral administration. Formulation as a suspension provides the phosphate derivatives of hydroxy chromans in a mixture with a dispersing or wetting agent, suspending agent, and one or more preservatives. Suitable dispersing or wetting agents are, for example, naturally-occurring phosphatides (e.g. lecithin or condensation products of ethylene oxide with a fatty acid, a long chain aliphatic alcohol or a partial ester derived from fatty acids) and a hexitol or a hexitol anhydride (e.g. polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

### Parenteral Compositions

The phosphate derivatives of hydroxy chromans may be administered parenterally by injection, infusion or implantation (intravenous, intramuscular, subcutaneous, or the like) in dosage forms, formulations or via suitable delivery devices or implants containing conventional, non-toxic pharmaceutically acceptable carriers. The formulation and preparation of such compositions is well known to those skilled in the art of pharmaceutical formulation. Specific formulations can be found in Remington: The Science and Practice of Pharmacy, supra.

Compositions for parenteral use may be presented in unit dosage forms (e.g. in single-dose ampoules) or in vials containing several doses and in which a suitable preservative may be added. The composition may be in form of a solution, a suspension, an emulsion, an infusion device or a delivery device for implantation or it may be presented as a dry powder to be reconstituted with water or another suitable vehicle before use. Apart from the phosphate derivatives of hydroxy chromans, the composition may include suitable parenterally acceptable carriers. The phosphate derivatives of hydroxy chromans may be incorporated into microspheres, microcapsules, nanoparticles, liposomes, or the like, for controlled release. Furthermore, the composition may include suspending, solubilizing, stabilizing, pH-adjusting agents and/or dispersing agents.

As indicated above, the pharmaceutical compositions may be in the form suitable for sterile injection. To prepare such a composition, the phosphate derivatives of hydroxy chromans are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives (e.g. methyl, ethyl or n-propyl p-hydroxybenzoate). In cases where the phosphate derivatives of hydroxy chromans are only sparingly or slightly soluble in water, a dissolution enhancing or solubilising agent can be added or the solvent may include 10-60% w/w of propylene glycol or the like.

### Rectal Compositions

For rectal application, suitable dosage forms for a composition include suppositories (emulsion or suspension type) and rectal gelatin capsules (solutions or suspensions). In a typical suppository formulation, the phosphate derivatives of hydroxy chromans are combined with an appropriate pharmaceutically acceptable suppository base such as cocoa butter, esterified fatty acids, glycerinated gelatin and various water-soluble or dispersible bases like polyethylene glycols and polyoxyethylene sorbitan fatty acid esters. Various additives, enhancers or surfactants may be incorporated.

### Vaginal Compositions

Compositions suitable for vaginal administration may be presented as pessaries, tampons, creams, gels, pastes, foams or sprays containing in addition to the phosphate derivatives of hydroxy chromans such carriers as are known in the art to be appropriate.

### Nasal and Inhalation Compositions

For administration to the respiratory tract, including intranasal administration, the phosphate derivatives of hydroxy chromans may be administered by any of the methods and formulations employed in the art for administration to the respiratory tract.

Thus in general the phosphate derivatives of hydroxy chromans may be administered in the form of a solution or a suspension or as a dry powder.

Solutions and suspensions will generally be aqueous, for example prepared from water alone (e.g. sterile or pyrogen-free water) or water and a physiologically acceptable co-solvent (e.g. ethanol, propylene glycol or polyethylene glycols such as PEG 400).

Such solutions or suspensions may additionally contain other excipients for example preservatives (such as benzalkonium chloride), solubilising agents/surfactants such as polysorbates (e.g. Tween 80, Span 80, benzalkonium chloride), buffering agents, isotonicity-adjusting agents (e.g. sodium chloride), absorption enhancers and viscosity enhancers. Suspensions may additionally contain suspending agents (e.g. microcrystalline cellulose and carboxymethyl cellulose sodium).

Solutions or suspensions are applied directly to the nasal cavity by conventional means, for example with a dropper, pipette or spray. The formulations may be provided in single or multidose form. In the latter case a means of dose metering is desirably provided. In the case of a dropper or pipette this may be achieved by the subject administering an appropriate, predetermined volume of the solution or suspension. In the case of a spray this may be achieved for example by means of a metering atomising spray pump.

Administration to the respiratory tract may also be achieved by means of an aerosol formulation in which the phosphate derivatives of hydroxy chromans are provided in a pressurised pack with a suitable propellant, such as a chlorofluorocarbon (CFC), for example dichlorodifluoromethane, trichlorofluoromethane or dichlorotetrafluoroethane, carbon dioxide or other suitable gas. The aerosol may conveniently also contain a surfactant such as lecithin. The dose of phosphate derivatives of hydroxy chromans may be controlled by provision of a metered valve.

Alternatively the phosphate derivatives of hydroxy chromans may be provided in the form of a dry powder, for example a powder mix of the compound in a suitable powder base such as lactose, starch, starch derivatives such as hydroxypropylmethyl cellulose and polyvinylpyrrolidine (PVP). Conveniently the powder carrier will form a gel in the nasal cavity. The powder composition may be presented in unit dose form, for example in capsules or cartridges of, for example, gelatin, or blister packs from which the powder may be administered by means of an inhaler.

In formulations intended for administration to the respiratory tract, including intranasal formulations, the phosphate derivatives of hydroxy chromans will generally have a small particle size, for example of the order of 5 microns or less. Such a particle size may be obtained by means known in the art, for example by micronisation.

When desired, formulations adapted to give sustained release of the phosphate derivatives of hydroxy chromans may be employed.

The phosphate derivatives of hydroxy chromans may be administered by oral inhalation as a free-flow powder via a “Diskhaler” (trade mark of Glaxo Wellcome plc or a meter dose aerosol inhaler.

### Topical Compositions

The pharmaceutical compositions may also be administered topically on the skin for percutaneous absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters and other kinds of transdermal drug delivery systems. The pharmaceutically acceptable carriers may include emulsifying agents, antioxidants, buffering agents, preservatives, humectants, penetration enhancers, chelating agents, gel forming agents, ointment bases, perfumes and skin protective agents.

Examples of emulsifying agents are naturally occurring gums (e.g. gum acacia, gum tragacanth) and naturally occurring phosphatides (e.g. soybean lecithin, sorbitan monooleate derivatives). Examples of antioxidants are butylated hydroxy anisole (BHA), ascorbic acid and derivatives thereof, butylated hydroxy anisole, and cysteine. Examples of preservatives are parabens, such as methyl or propyl p-hydroxybenzoate and benzalonium chloride. Examples of humectants are glycerin, propylene glycol, sorbitol and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol and Azone®. Examples of chelating agents are sodium EDTA, citric acid and phosphoric acid. Examples of gel forming agents are Carbopol, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols and condensation products between sorbitan esters of fatty acids and ethylene oxide (e.g. polyoxyethylene sorbitan monooleate (Tween)).

The pharmaceutical compositions described above for topical administration on the skin may also be used in connection with topical administration onto or close to the part of the body that is to be treated. The compositions may be adapted for direct application or for introduction into relevant orifice(s) of the body (e.g. rectal, urethral, vaginal or oral orifices). The composition may be applied by means of special delivery devices such as dressings or alternatively plasters, pads, sponges, strips or other forms of suitable flexible material.

### Ocular Compositions

For application to the eye, the phosphate derivatives of hydroxy chromans may be in the form of a solution or suspension in a suitable sterile aqueous or non-aqueous vehicle. Additives, for instance buffers, preservatives including bactericidal and fungicidal agents, such as phenyl mercuric acetate or nitrate, benzalkonium chloride, or chlorohexidine and thickening agents such as hypromellose may also be included.

### Veterinary Compositions

The phosphate derivatives of hydroxy chromans may also be presented for use in the form of veterinary compositions, which may be prepared, for example, by methods that are conventional in the art. Examples of such veterinary compositions include those adapted for:

(a) oral administration, external application, for example drenches (e.g. aqueous or non-aqueous solutions or suspensions); tablets or boluses; powders, granules or pellets for admixture with feed stuffs; pastes for application to the tongue;

(b) parenteral administration for example by subcutaneous, intramuscular or intravenous injection, e.g. as a sterile solution or suspension; or (when appropriate) by intramammary injection where a suspension or solution is introduced in the udder via the teat;

(c) topical applications, for example, as a cream, ointment or spray applied to the skin; or

(d) rectally or intravaginally, for example, as a pessary, cream or foam.

### Methods of Treatment or Prophylaxis

The phosphate derivatives of hydroxy chromans may be used in the treatment and/or prophylaxis of immune disorders, inflammatory disorders, and/or cellular proliferative disorders.

Generally, the terms “treatment” and “prophylaxis” mean affecting a subject, tissue or cell to obtain a desired pharmacological and/or physiological effect and include: (a) preventing the disorder from occurring in a subject that may be predisposed to the disorder, but has not yet been diagnosed as having it; (b) inhibiting the disorder, i.e. arresting its development; or (c) relieving or ameliorating the effects of the disorder, i.e. cause regression of the effects of the disorder.

The term “subject” as used herein refers to any animal having a disorder which requires treatment and/or prophylaxis with a pharmaceutically-active agent. The subject may be an animal, such as a mammal, preferably a human, or may be a non-human primate or non-primates such as in animal model testing. While it is particularly contemplated that the phosphate derivatives of hydroxy chromans are suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates.

The term “immune disorders” and like terms means a deficiency, disease, disorder or condition caused by the immune system of a subject (e.g. human or non-human animal), including autoimmune disorders. Immune disorders include those deficiencies, diseases, disorders or conditions that have an immune component and those that are substantially or entirely immune system-mediated. Autoimmune disorders are those wherein the subject's own immune system mistakenly attacks itself, thereby targeting the cells, tissues, and/or organs of the subject's own body. For example, the autoimmune reaction is directed against the nervous system in multiple sclerosis and the gut in Crohn's disease. In other autoimmune disorders such as systemic lupus erythematosus (lupus), affected tissues and organs may vary among individuals with the same disease. One subject with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus. Specific autoimmune disorders that may be ameliorated include without limitation, autoimmune disorders of the nervous system (e.g. multiple sclerosis, myasthenia gravis, autoimmune neuropathies such as Guillain-Barre, and autoimmune uveitis), autoimmune disorders of the blood (e.g. autoimmune hemolytic anemia, pernicious anemia, and autoimmune thrombocytopenia), autoimmune disorders of the blood vessels (e.g. temporal arteritis, anti-phospholipid syndrome, vasculitides such as Wegener's granulomatosis, and Behcet's disease), autoimmune disorders of the skin (e.g. psoriasis, dermatitis herpetiformis, pemphigus vulgaris, and vitiligo), autoimmune disorders of the gastrointestinal system (e.g. Crohn's disease, ulcerative colitis, primary biliary cirrhosis, and autoimmune hepatitis), autoimmune disorders of the endocrine glands (e.g. Type 1 or immune-mediated diabetes mellitus), Grave's disease, Hashimoto's thyroiditis, autoimmune oophoritis and orchitis, autoimmune disorders of the adrenal gland, autoimmune disorders of multiple organs including connective tissue and musculoskeletal system diseases (e.g. rheumatoid arthritis, systemic lupus erythematosus, scleroderma, polymyositis, dermatomyositis, spondyloarthropathies such as ankylosing spondylitis, and Sjogren's syndrome). In addition, other immune system mediated diseases, such as graft-versus-host disease, and allergic disorders including asthma and anaphylaxis, are also included in the definition of immune disorders herein. Because a number of immune disorders are caused by inflammation, there is some overlap between disorders that are considered immune disorders and inflammatory disorders. For the purpose of this invention, in the case of such an overlapping disorder, it is to be considered an immune disorder.

Immune deficiencies usually result from an impaired immune system and can leave the body vulnerable to various viral, bacterial, or fungal opportunitistic infections. Causes of some immune deficiencies include various viral illnesses, chronic illness, or immune system illnesses (especially HIV/AIDS).

The term “inflammatory disorder” and like terms means a disease, disorder or condition characterized by inflammation of body tissue or having an inflammatory component. These include local inflammatory responses and systemic inflammation. Examples of such inflammatory disorders include: transplant rejection, including skin graft rejection; chronic inflammatory disorders of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome, and chronic obstructive airway disease; inflammatory disorders of the eye including corneal dystrophy, trachoma, onchocerciasis, uveitis, sympathetic ophthalmitis and endophthalmitis; chronic inflammatory disorders of the gums, including gingivitis and periodontitis; tuberculosis; leprosy; inflammatory diseases of the kidney including uremic complications, glomerulonephritis and nephrosis; inflammatory disorders of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune disorders, immune-complex vasculitis, systemic lupus and erythematodes; systemic lupus erythematosus (SLE); and inflammatory diseases of the heart such as cardiomyopathy, ischemic heart disease hypercholesterolemia, atherosclerosis; as well as various other diseases with significant inflammatory components, including preeclampsia, chronic liver failure, brain and spinal cord trauma, and cancer. There may also be a systemic inflammation of the body, exemplified by gram-positive or gram negative shock, hemorrhagic or anaphylactic shock, or shock induced by cancer chemotherapy in response to pro-inflammatory cytokines, for example, shock associated with pro-inflammatory cytokines. Such shock can be induced, for example, by a chemotherapeutic agent used in cancer chemotherapy.

The term “cellular proliferative disorder” refers to any cellular disorder in which the cells proliferate more rapidly than normal tissue growth. Cellular proliferative disorders, includes but are not limited to, neoplasms. A neoplasm is an abnormal tissue growth, generally forming a distinct mass that grows by cellular proliferation more rapidly than normal tissue growth. Neoplasms show partial or total lack of structural organisation and functional coordination with normal tissue. These can be broadly classified into three major types. Malignant neoplasms arising from epithelial structures called carcinomas, malignant neoplasms that originate from connective tissues such as muscle, cartilage, fat or bone are called sarcomas and malignant tumours affecting hematopoietic structures (structures pertaining to the formation of blood cells) including components of the immune system called leukaemias and lymphomas. A tumour is the neoplastic growth of the disease cancer. As used herein, a “neoplasm”, also referred to as a “tumour” is intended to encompass hematopoitic neoplasms as well as solid neoplasms. Other cellular proliferative disorders include, but are not limited to arthritis, graft rejection, inflammatory bowel disease, proliferation induced after medical procedures, including, but not limited to, surgery, angioplasty, and the like.

The phosphate derivatives of hydroxy chromans are particularly useful for the treatment and/or prophylaxis of cancer including solid tumours such as skin, breast, brain, cervical carcinomas, testicular carcinomas, and so on. More particularly, cancers that may be treated by the phosphate derivatives of hydroxy compounds of the invention include, but are not limited to: Cardiac: sarcoma (angiosarcoma, fibrosarcoma, rhabdomyosarcoma. Liposarcoma), myxoma, rhabdomyoma, fibroma, lipoma and teratoma; Lung: bronchogenic carcinoma (squamous cell, undifferentiatied small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma; lymphoma, leiomyosarcoma), pancreas (ductal adenocarcinoma, insulinoma, glucagonoma, gastrinoma, carcinoid tumours, vipoma), small bowel (adenocarcinoma, lymphoma, carcinoid tumours, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumour (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumours, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumour chordoma, osteochronfroma (osteocartilaginous exostoses), benign chondroma, chondroblastoma, chondromyxofibroma, osteoid osteoma and giant cell tumours; Nervous system: skull (osteoma, hemangioma, granuloma, xanthoma, osteitis deformans), meninges (meningioma, meningiosarcoma, gliomatosis); brain (astrocytoma, medulloblastoma, glioma, ependymoma, germinoma (pinealoma), glioblastoma multiform, oligodendroglioma, schwannoma, retinoblastoma, congenital tumours), spinal cord neurofibroma, meningioma, glioma, sarcoma); Gynecological: uterus (endometrial carcinoma), cervix (cervical carcinoma, pre-tumour cervical dysplasia), ovaries (ovarian carcinoma (serous cystadenocarcinoma, mucinous cystadenocarcinoma, unclassified carcinoma), granulosa-thecal cell tumours, Sertoli-Leydig cell tumours, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands; neuroblastoma.

### Dosages

The term “therapeutically effective amount” means an amount of phosphate derivatives of hydroxy chromans effective to yield a desired therapeutic response.

It will be understood that the specific dose level for any particular subject will depend upon a variety of factors including the activity of the specific phosphate derivatives of hydroxy chromans employed, the age, body weight, general health, sex, diet, time of administration, route of administration, rate of excretion, type of formulation, and the severity of the particular disorder undergoing therapy.

The amount of phosphate derivatives of hydroxy chromans that may be combined with the carrier materials to produce a single dosage will also vary depending upon the subject treated and the particular mode of administration. For example, a formulation intended for oral administration to humans may contain about 5 mg to 2 g of the phosphate derivatives of hydroxy chromans with an appropriate and convenient amount of carrier material which may vary from about 5 to 95% of the total composition. Dosage unit forms will generally contain between from about 5 mg to 500 mg of the phosphate derivatives of hydroxy chromans.

In one embodiment, the dosage level for an alpha-tocopheryl phosphate mixture (alpha-TPm) comprising mono-tocopheryl phosphate (TP) and di-tocopheryl phosphate (T2P) in a ratio of 2:1 for administration to a human may be of the order of about 0.5 mg to about 100 mg per kilogram body weight, with a preferred dosage range between about 0.5 mg to about 30 mg per kilogram body weight per day (from about 0.5 gms to about 2 gms per patient per day).

### DETAILED DESCRIPTION OF DRAWINGS

In the Examples which follow, reference will be made to the accompanying drawings in which:

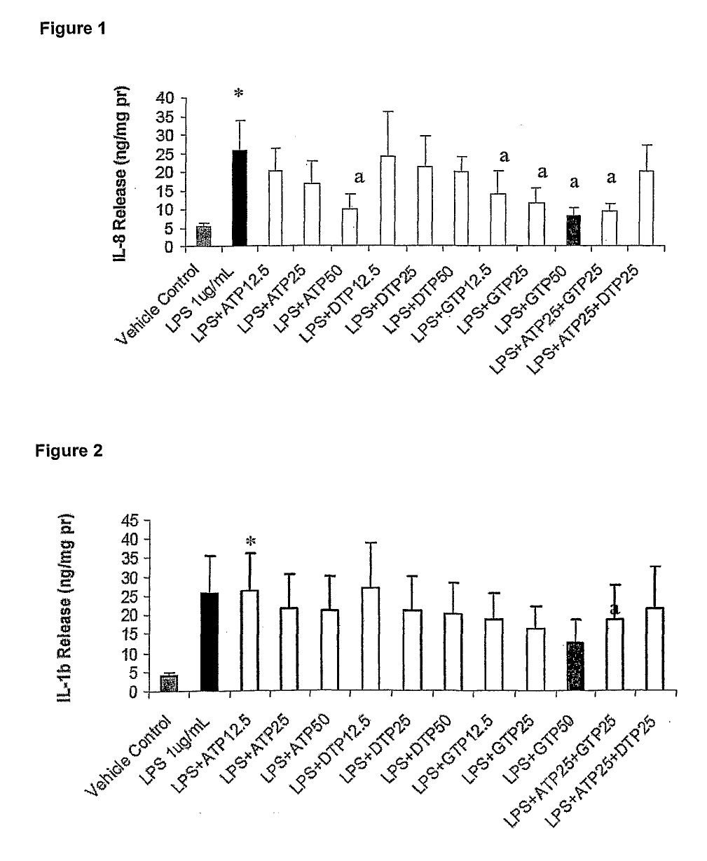

FIG. 1 is a graph showing the effect of tocopheryl phosphate mixtures on IL-8 release from human monocytes (n=5) from Example 1. In this figure, * indicates p<0.01 compared to the vehicle control; and ^{a} indicates p<0.05 compared to LPS;

FIG. 2 is a graph showing the effect of tocopheryl phosphate mixtures on IL-1β release from human monocytes (n=5) from Example 1. In this figure, * indicates p<0.01 compared to the vehicle control; and ^{a} indicates p<0.05 compared to LPS;

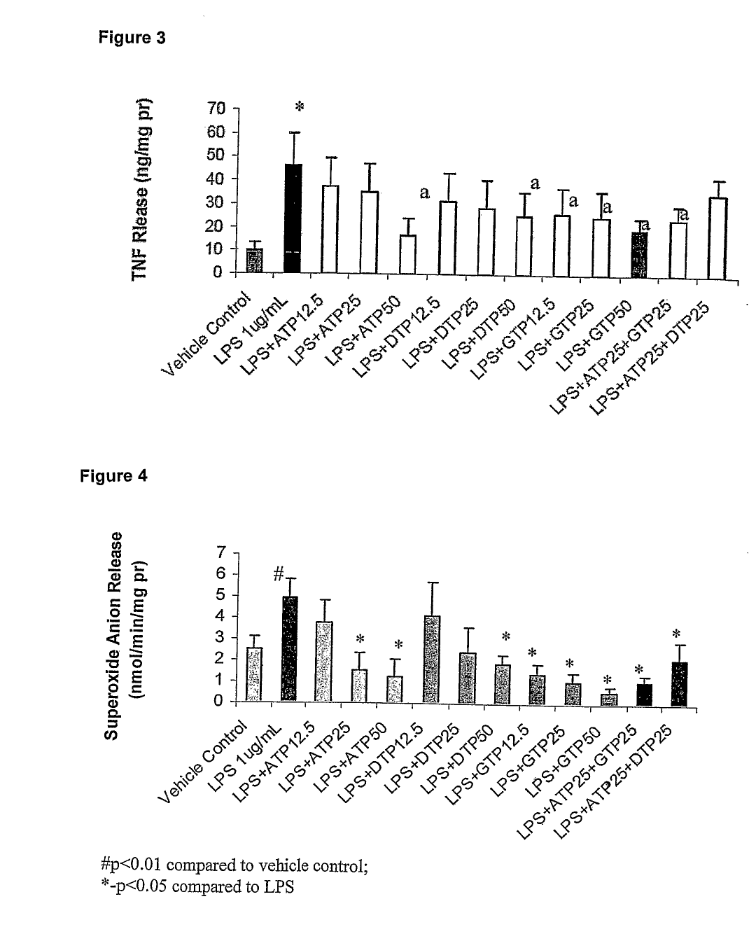

FIG. 3 is a graph showing the effect of tocopheryl phosphate mixtures on TNFα release from human monocytes (n=5) from Example 1. In this figure, * indicates p<0.01 compared to the vehicle control; and ^{a} indicates p<0.05 compared to LPS;

FIG. 4 is a graph showing the effect of tocopheryl phosphate mixtures on superoxide anion release from human monocytes (n=5) from Example 1. In this figure, # indicates p<0.01 compared to the vehicle control; * indicates p<0.05 compared to LPS;

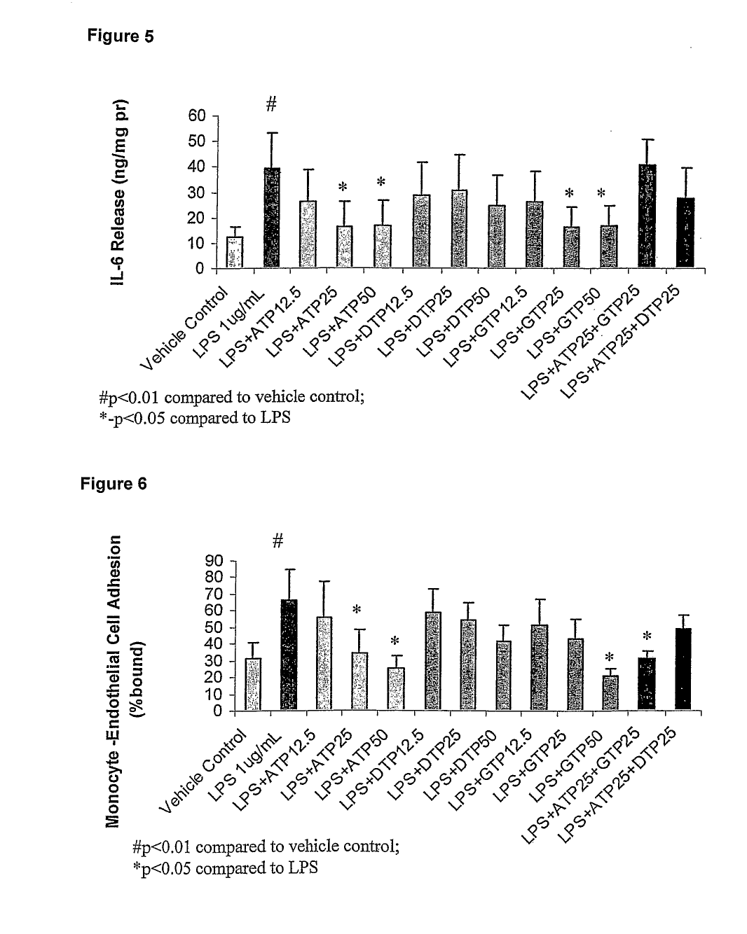

FIG. 5 is a graph showing the effect of tocopheryl phosphate mixtures on IL-6 release from human monocytes (n=5) from Example 1. In this figure, # indicates p<0.01 compared to the vehicle control; and LPS+ATP25, LPS+ATP50, LPS+DTP50, LPS+GTP12.5, LPS+GTP25, LPS+GTP50, LPS+ATP25+GTP25, LPS+ATP25+DTP25 are significantly different compared to LPS (p<0.05);

FIG. 6 is a graph showing the effect of tocopheryl phosphate mixtures on monocyte-endothelial cell adhesion (n=5) from Example 1. In this figure, # indicates p<0.01 compared to the vehicle control; and LPS+ATP25, LPS+ATP50, LPS+GTP50, LPS+ATP25+DTP25 are significantly different compared to LPS (P<0.05);

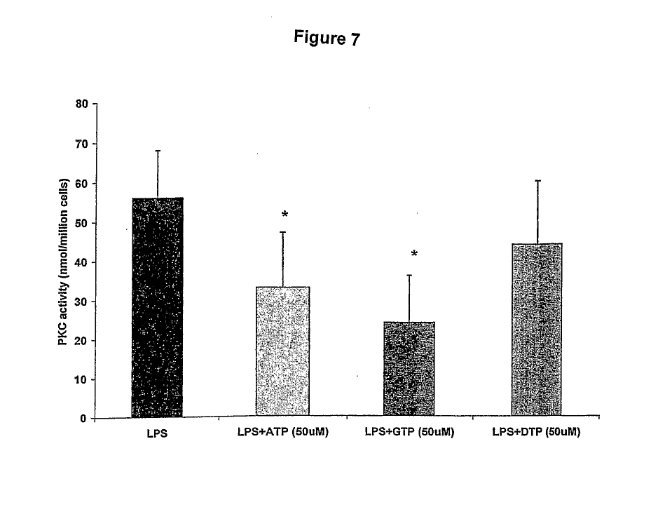

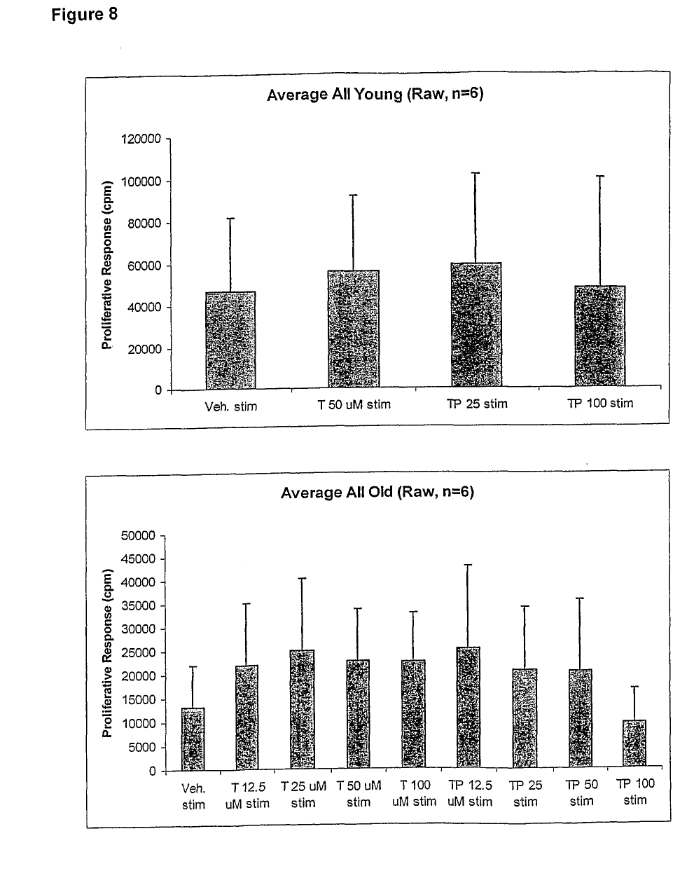

FIG. 7 is a graph showing the effect of tocopheryl phosphate mixtures on monocyte PKC activity (n=3) from Example 1;

FIG. 8 is a graph showing the averages of proliferative responses (mean +sd) of six young and six old mice from Example 2;

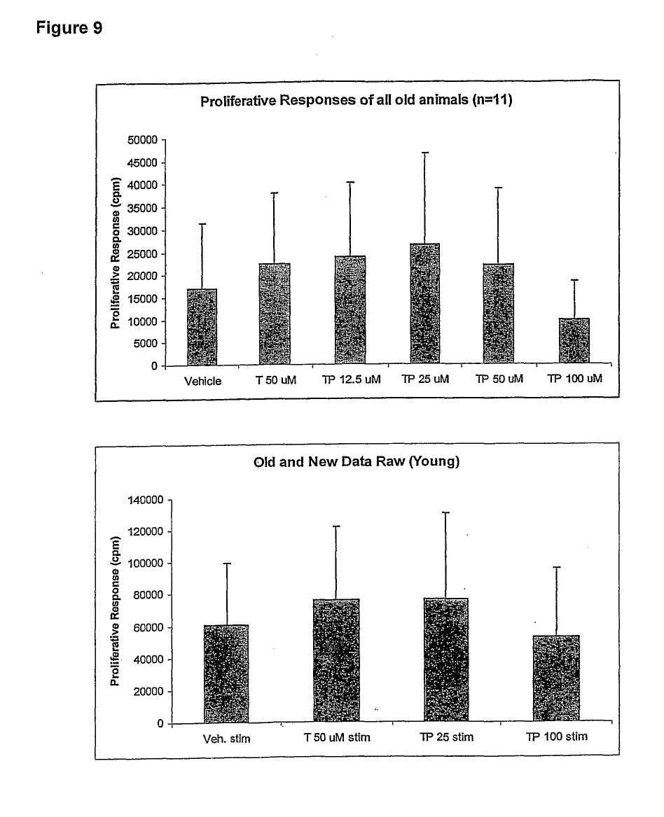

FIG. 9 is a graph showing the average of old and new proliferative responses from young and old mice (n=11) from Example 2;

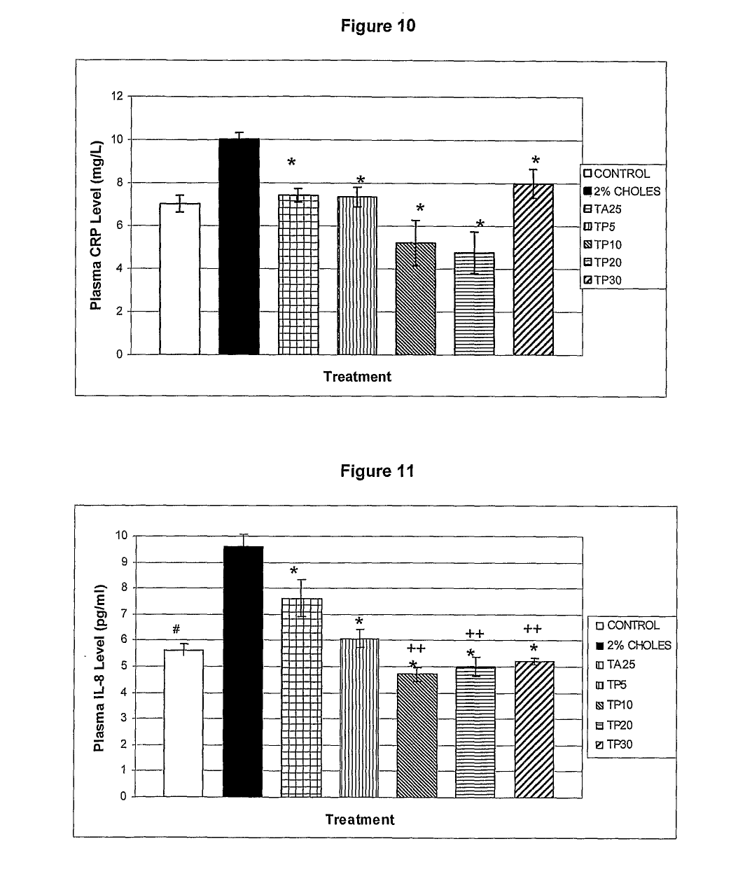

FIG. 10 is a graph showing the effect of an α-tocopheryl phosphate mixture (alpha-TPm) on CRP levels in the plasma of hypercholesterolemic rabbits (n=5-8). In this figure, * indicates P<0.05 2% cholesterol fed animals compared to the various treatments;

FIG. 11 is a graph showing the effect of an α-tocopheryl phosphate mixture (alpha-TPm) on IL-8 levels in the plasma of hypercholesterolemic rabbits (n=5-8). In this figure, # indicates P<0.05 control compared to 2% cholesterol treatment; * indicates P<0.05 2% cholesterol fed animals compared to the various treatments; and ++ indicates P<0.05 ta25 compared to TPm treatments;

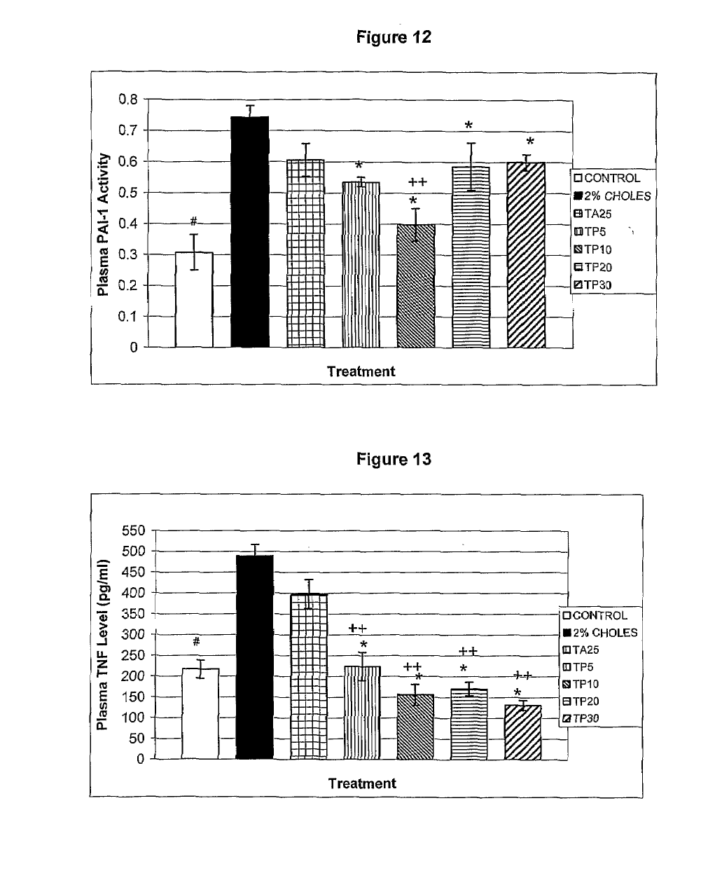

FIG. 12 is a graph showing the effect of an α-tocopheryl phosphate mixture (alpha-TPm) on PAI-1 activity in the plasma of hypercholesterolemic rabbits (n=5-8). In this figure, # indicates P<0.05 control compared to 2% cholesterol treatment; * indicates P<0.05 2% cholesterol fed animals compared to the various treatments; and ++ indicates P<0.05 TA25 compared to TPm treatments;

FIG. 13 is a graph showing the effect of an α-tocopheryl phosphate mixture (alpha-TPm) on TNF level in the plasma of hypercholesterolemic rabbits (n=5-8). In this figure, # indicates P<0.05 Control compared to 2% cholesterol treatment; * indicates P<0.05 2% cholesterol fed animals compared to the various treatments; and ++ indicates P<0.05 TA25 compared to TPm treatments;

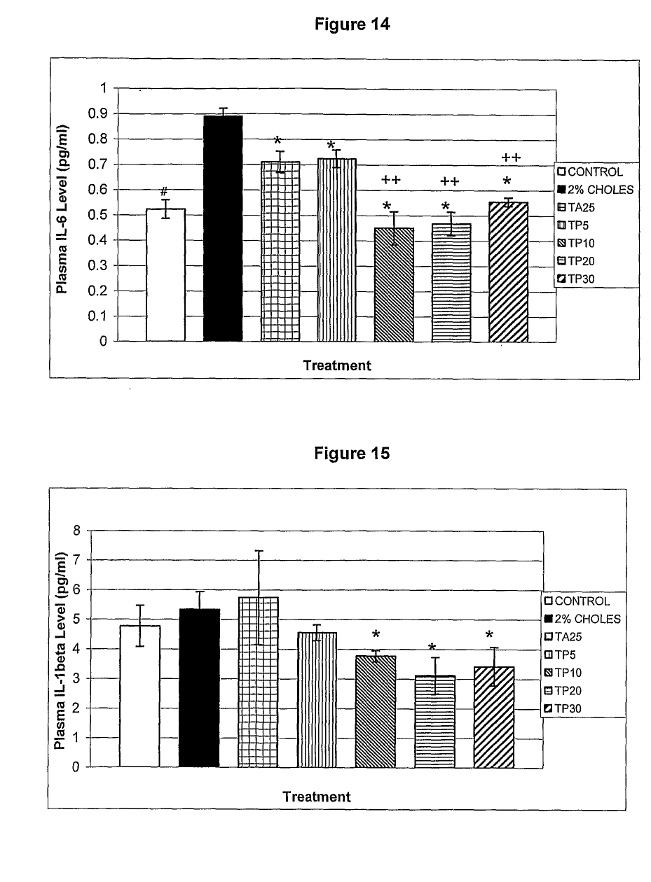

FIG. 14 is a graph showing the effect of an α-tocopheryl phosphate mixture (alpha-TPm) on IL-6 level in the plasma of hypercholesterolemic rabbits (n=5-8). In this figure, # indicates P<0.05 Control compared to 2% cholesterol treatment; * indicates P<0.05 2% cholesterol fed animals compared to the various treatments; and ++ indicates P<0.05 TA25 compared to TPm treatments.

FIG. 15 is a graph showing the effect of an α-tocopheryl phosphate mixture (alpha-TPm) on IL-1β level in the plasma of hypercholesterolemic rabbits (n=5-8). In this figure, * indicates P<0.05 2% cholesterol fed animals compared to the various treatments;

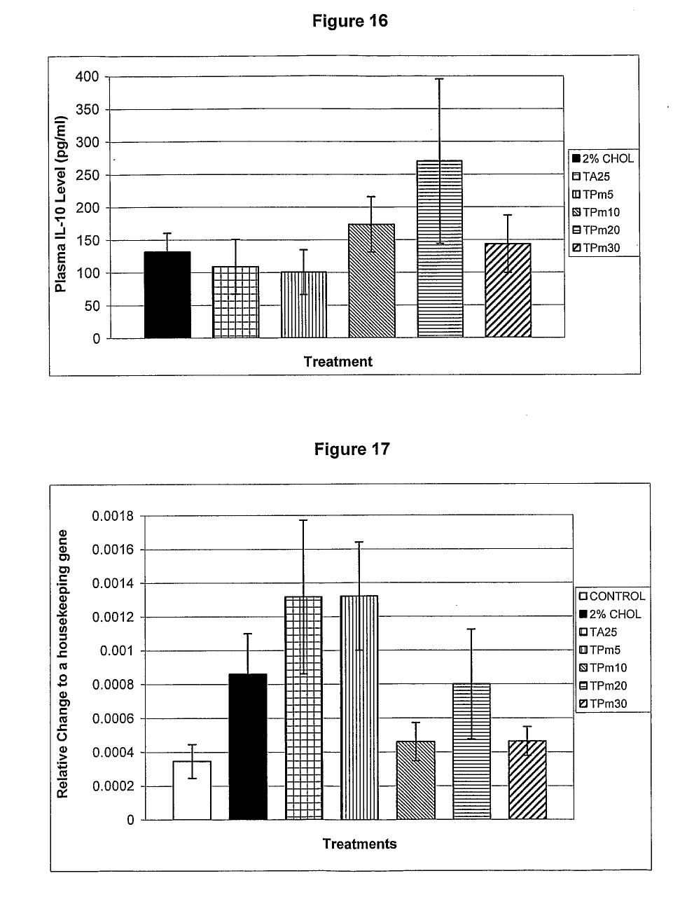

FIG. 16 is a graph showing the effect of an α-tocopheryl phosphate mixture (alpha-TPm) on IL-10 level in the plasma of hypercholesterolemic rabbits (n=5-8). In this figure, * indicates P<0.05 2% cholesterol fed animals compared to the various treatments; and

FIG. 17 is a graph showing the effect of an α-tocopheryl phosphate mixture (alpha-TPm) on CD36 expression in the aortas of hypercholesterolemic rabbits (n=5-8). In this figure, * indicates P<0.05 2% cholesterol fed animals compared to the various treatments.

### EXAMPLES

The invention will now be further described with reference to the following non-limiting Examples.

### Example 1

This example investigates the effect of tocopheryl phosphate mixtures (TPm) on the release of IL-8, IL-1β, TNFα and IL-6, on superoxide anion release, on monocyte-endothelial cell adhesion, and on monocyte PKC activity.

### Materials:

| LPS | Lipopolysaccharide derived from Bacteroides fragilis |
|---|---|
| | is commonly used in these in vitro studies. It acts |
| | as a cell stimulant. |
| ATP | Alpha-tocopheryl phosphate mixture (alpha-TPm) |
| | comprising mono-tocopheryl phosphate and di- |
| | tocopheryl phosphate in a ratio of approximately |
| | 2:1. |
| DTP | Delta-tocopheryl phosphate mixture (delta-TPm) |
| | comprising mono-tocopheryl phosphate and di- |
| | tocopheryl phosphate in a ratio of approximately |
| | 2:1. |
| GTP | Gamma-tocopheryl phosphate mixture (gamma-TPm) |
| | comprising mono-tocopheryl phosphate and di- |
| | tocopheryl phosphate in a ratio of approximately |
| | 2:1. |

### Methods:

Monocyte Isolation: Following informed consent, fasting blood in heparin anticoagulated tubes was obtained from normal healthy volunteers. Peripheral blood mononuclear cells were obtained after layering the blood carefully on a Ficoll Hypaque gradient. After 2 washes, monocytes were isolated by negative magnetic separation using the MACS reagents from Miltenyi Biotech. Cells were resuspended at 1×10⁶ cells/ml.

Cells were pre-incubated with alpha, delta or gamma tocopheryl phosphate mixtures or the combination or vehicle control as denoted in the figures for 24 hours prior to being activated with LPS for 1 hour for assessment of superoxide anion release, 8 hours for cytokine and chemokine release and for 4 hours for monocyte-endothelial cell adhesion.

Measurement of superoxide anion release was performed using SOD-inhibitable ferricytochrome C reduction and expressed as n moles per minute per mg protein. Cytokine (IL-1β & TNFα) and chemokine (IL-8) release from monocytes was performed in supernatants of treated monocytes using specific fluorescent labelled antibodies on the BDFACS Array and expressed per mg cell protein. For monocyte-endothelial cells adhesion, Human aortic endothelial cells were used between passages 2-5. Treated and control monocytes were labelled with CFDA-SE for 1 hour at 37° C. following by washing to remove unbound dye. The labelled monocytes were then added on the HAEC and incubated for an additional hour at 37° C. Cells were then washed and the fluorescence quantitated by excitation at 485 and emission at 535 nm and expressed as % bound compared to the total.

Statistical Analyses: All data are expressed as mean+/−SD of 5 experiments in duplicate and statistical analyses were performed using GraphPad Prizm software. Following ANOVA, parametric data were analysed using paired t-tests and non-parametric data using Wilcoxon signed rank tests and p<0.05 was considered significant.

### Results:

The results are set out in FIGS. 1 to 7. In FIGS. 1 to 3, * means p<0.01 compared to vehicle control and ^{a} means p<0.05 compared to LPS. In FIGS. 4 to 6, # means p<0.01 compared to vehicle control and * means p<0.05 compared to LPS.

LPS-activated IL-8 release was significantly inhibited with GTP at 12.5 μg/ml, 25 μg/ml and 50 μg/ml (FIG. 1). LPS-activated IL-8 release was also defectively inhibited by DTP, ATP and the combination of GTP with ATP.

FIG. 2 shows LPS-activated IL-1β release was significantly inhibited with GTP at 50 μg/ml.

FIG. 3 shows LPS-activated TNFα release was significantly inhibited with GTP at 12.5 μg/ml, 25 μg/ml and 5 μg/ml, and by ATP, DTP, and the combination of ATP with GTP.

FIG. 4 shows the effect of tocopheryl phosphate mixtures on superoxide anion release from human monocytes (n=5).

FIG. 5 shows the effect of tocopheryl phosphate mixtures on IL-6 release from human monocytes (n=5) and LPS+ATP25, LPS+ATP50, LPS+DTP50, LPS+GTP12.5, LPS+GTP25, LPS+GTP50, LPS+ATP25+GTP25, LPS+ATP25+DTP25 are significantly different compared to LPS (pc0.05).

FIG. 6 shows the effect of tocopheryl phosphate mixtures on monocyte-endothelial cell adhesion (n=5) and LPS+ATP25, LPS+ATP50, LPS+GTP50, LPS+ATP25+DTP25 are significantly different compared to LPS (p<0.05).

FIG. 7 shows the effect of tocopheryl phosphate mixtures on monocyte PKC activity (n=3).

### Discussion:

Adhesion of monocytes to LPS-activated HAEC was significantly inhibited with ATP (>25 μg/ml, 65%, p<0.05) and with GTP (50 μg/ml, 71%, p<0.05). These monocytes play a very important role in immune response mechanisms.

ATP, DTP and GTP significantly decreased PKC activity in activated monocytes (50 μg/ml; 44%, 21% and 56% respectively, p<0.05).

Monocyte LPS-activated superoxide anion release was significantly inhibited with ATP (≧25 μg/ml, 75%, p<0.05), DTP (50 μg/ml, 61%, p<0.05) and GTP (≧12.5 μg/ml, 75%, p<0.05).

Monocyte LPS-activated IL-8 release was significantly inhibited with ATP (50 μg/ml, 65%, p<0.05), and with GTP (≧12.5 μg/ml, 61%, p<0.05).

LPS-activated IL-1β release was significantly inhibited only with GTP (50 μg/ml, 43%, p<0.05), IL-6 release with ATP (≧25 μg/ml, 46%, p<0.05) and GTP (≧25 μg/ml, 41%, p<0.05) and TNFα release was significantly inhibited with ATP (50 μg/ml, 59%, p<0.05), DTP (50 μg/ml, 44%, p<0.05), and GTP (≧12.5 μg/ml, 48%, p<0.05).

### Conclusion:

All of the biomarkers analysed play an important role in regulating the immune system. The results show that the tocopheryl phosphate mixtures were able to significantly affect the functions of these markers at various concentrations.

ATP and GTP appear to exert anti-inflammatory and immune modulatory effects in monocytes with GTP being superior to ATP.

GTP appears to exert the most anti-inflammatory and immune related effects in monocytes and this appears to be mediated via inhibition of PKC activation.

The data suggests that tocopheryl phosphates are effective in the modulation of the cytokines IL-8, IL-1β, IL-6 and TNFα.

### Example 2

This study investigated the effect of an alpha-tocopheryl phosphate mixture (ATP) on T cell proliferation. T cell proliferation is modulated by cytokines.

### Methodology:

T cells from young (4 to 6 months) and old (>22 months) C57BL mice were isolated and purified from splenocytes by negative selection using Pan T cell isolation kit from Miltenyi Biotech.

The tocopherol phosphate mixture (alpha-TP) provided by Phosphagenics Limited was composed of approximately ⅔ mono-tocopheryl phosphate (MW 510.7) and ⅓ di-tocopheryl phosphate (MW 923.3), which makes the MW of the compound 598.0. Ethanol was used to dissolve the solid alpha-TP mixture to make a 107.52 mg/ml stock solution (158.11 mM). 34 mg/ml alpha-tocopherol (alpha-T) in ethanol was used as a stock solution of alpha-T (79.06 mM). The solutions used for pre-incubation and incubation were prepared as follows:

58.8 μl of the following solutions was added to 440.6 μl FBS: 100% ethanol (vehicle control): alpha-T stock (34 mg/ml), alpha-T solutions (53.76 mg/ml), alpha-T solution (26.88 mg/ml), alpha-T solution (13.44 mg/ml), alpha-TP solution (107.52 mg/ml), alpha-TP solution (53.76 mg/mL), alpha-TP solution (26.88 mg/mL), and alpha-TP solution (13.44 mg/mL).

In order to incorporate alpha-T or alpha-TP into the solutions well, they were vortexed and placed in 37° C. water bath for five minutes and this was repeated twice more for a total of 15 minutes incubation.

172 μl of each of the above solutions were added to 7.180 ml of RPMI (+add) and 648 μl FBS to make 10% FBS containing RPMI with vehicle control, alpha-T (86 mg/ml, 200 μM), alpha-T (43 mg/ml, 100 μM), alpha-T (21.5 mg/ml, 50 μM), alpha-T (10.75 mg/ml, 25 μM), alpha-T (136.09 μg/ml, 200 μM), alpha-TP (68.04 μg/ml, 100 μM), alpha-TP (34.02 μg/ml, 50 μM), or alpha-TP (17.01 μg/ml, 25 μM).

The T cells (1.5×10⁶ cells/treatment) were pre-incubated for 4 hours with vehicle control (ethanol), alpha-T (12.5 μM, 25 μM, 50 μM, 100 μM), or alpha-TP (12.5 μM, 25 μM, 50 μM, 100 μM) at 35° C. at 2×10⁶ cells/ml.

After pre-incubation, the cells were stimulated (2×10⁵ cells/well) in triplicate in a round bottom 96-well plate with plate-bound anti-CD3 (5 μg/ml, anti-CD3e (145-2C11), Pharmingen Cat# 553058) and soluble anti-CD28 (2 μg/ml, final concentration, anti-CD28 (37.51), Pharmingen Cat# 553294) in RPMI medium containing 5% FBS (final concentration and same concentration of vehicle, alpha-tocopherol or alpha-tocopherol phosphate used in the pre-incubation. The cells were incubated at 35° C., 5% CO₂ for 64 hours, then, pulsed with 0.5 μCi of [³H] thymidine for 8 hours. The cells were harvested onto filter paper and proliferation was quantified as the amount of [³H] Thymidine incorporated into DNA, as determined by liquid scintillation counting (Beckman Counter, Fullerton, Calif.).

The results presented are from 5 separate experiments with n=6.

### Results:

Fisher's Least-Significant-Difference Test indicates a significant increase in proliferation with the alpha-TP 12.5 μM treatment (p=0.010) and all the alpha-T treatments compared to vehicle in the old mice (p<0.05).

FIG. 8 shows the averages of proliferative responses (mean +sd) of six young and six old mice.

FIG. 9 shows the average of old and new proliferative responses from young and old mice (n=11). For these results, the five young and five old mice from the previous experiment were added onto the six young and old mice from the revised protocol. In the old mice, compared to the vehicle treatment, the alpha-TP 25 μM treatment significantly increased T cell proliferation (p=0.011), while the alpha-TP 100 μM treatment significantly inhibited proliferation (p=0.029). However, in the young mice no significant difference was found among the treatments.

### Conclusion:

In these experiments, 12.5 μM alpha-TP elicited the highest proliferative response among the alpha-TP treatments, while all the alpha-T treatments resulted in significantly higher proliferative response compared with the vehicle control, as shown in FIG. 8.

When all the data from the experiments were combined, results indicate that in vitro treatment of 25 μM alpha-TP elicits the highest proliferative response in old T cells, which substantiates our previous findings from our preliminary experiment with n=5 in each group.

The results of the experiments described here show that the TP mixtures can modulate cytokine activity and/or expression, and are more effective than non-phosphorylated tocopherol at eliciting T cell proliferation, and hence are more potent immuno-enhancers.

### Example 3

This study investigated the effect of oral administration of an alpha-tocopheryl phosphate mixture (alpha-TPm) in hypercholesterolemic rabbits (fed a 2% cholesterol diet). Plasma assessment of the level of pro-inflammatory cytokines (e.g. IL-1β, IL-6, IL-8, TNF) and anti-inflammatory cytokines (e.g. IL-10) and other inflammatory biomarkers (e.g. CRP) to assess the level of inflammation induced by a diet high in cholesterol and the benefits provided by the alpha-tocopheryl phosphate mixture.

### Methodology:

47 female New Zealand albino rabbits were obtained at 4-8 weeks of age. They were divided into 7 treatment groups, which consisted of 5-10 animals per group. All the animals were placed on a vitamin E stripped diet, and all the diets contained 2% cholesterol except for the control group (which contained no cholesterol) for 4 weeks (containing the various TA or TPm treatments outlined below).

| Treatment | Actual Average Dose | | TPm added per kg of | | |
|---|---|---|---|---|---|
| Group | Delivered | | rodent feed | | n |
| Control | 0 | mg/kg body weight | 0 | mg | 10 |
| TA25 | 22.6 | mg/kg body weight | 300 | mg | 6 |
| TPm5 | 4.6 | mg/kg body weight | 60 | mg | 6 |
| TPm10 | 9.1 | mg/kg body weight | 120 | mg | 5 |
| TPm20 | 18.4 | mg/kg body weight | 240 | mg | 5 |
| TPm30 | 33.7 | mg/kg body weight | 360 | mg | 7 |

(For control normal diet rabbits n = 8).

At the end of the treatment period the animals were sacrificed and the blood collected. The plasma was obtained from this blood and the levels of various markers of inflammation were determined and included: IL-1β, IL-6, IL-8, TNF, PAI-1, and CRP. Other markers of inflammation such as CD36 was determined from mRNA isolated from the aortas of the rabbits to determine expression levels.

### Results:

FIGS. 10 to 15 show the plasma level of a number of pro-inflammatory cytokines that have all been elevated due to the addition of a 2% cholesterol diet compared to no cholesterol fed rabbits (indicated by #). These elevated pro-inflammatory cytokines indicate an environment prone to inflammatory diseases. Treatment of these hypercholesterolemic rabbits with tocopherol acetate (TA), at 300 mg/kg feed, showed modest decreases in these pro-inflammatory markers, three of which were considered significant (CRP, IL-8, and IL-6). While TPm showed significant reductions in all the pro-inflammatory cytokines tested (indicated by *), as well as significant reduction compared to TA treated rabbits (indicated by ++).

FIG. 17 shows the aortic CD36 expression, relative to a housekeeping gene, of these hypercholesterolemic rabbits treated with the various compounds. As a pro-inflammatory marker, again the high cholesterol diet was shown to significantly elevate the level of CD36 expression in these rabbits. Treatment with TPm showed a decrease in CD36 expression.

### Conclusion:

In these experiments, TPm treatment significantly decreased the expression of a number of pro-inflammatory cytokines and markers, including: IL-1β, IL-6, IL-8, CRP, TNF, CRP, and CD36. TPm treatment also increased the expression of anti-inflammatory cytokines and increase in anti-inflammatory cytokines indicates a balanced environment in which the overall inflammatory condition produced by the high cholesterol diet is reduced, in some cases back to no cholesterol control diet levels. The 3 highest doses tested that is 120, 240 and 360 mg TPm/kg feed, in particular, showed the greatest decrease in these cytokines and markers.

In the subject specification, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

It must be noted that, as used in the subject specification, the singular forms “a”, “an” and “the” include plural aspects unless the context clearly dictates otherwise.

It will be understood to persons skilled in the art of the invention that many modifications may be made to the invention without departing from the spirit and scope of the invention.

1. A method of modulating one or more immuno-regulatory cytokines comprising administering to a subject a therapeutically effective amount of one or more phosphate derivatives of one or more hydroxy chromans, or complexes thereof.

2. The method according to claim 1, wherein the immuno-regulatory cytokine is a pro-inflammatory cytokine or anti-inflammatory cytokine.

3. The method according to claim 2, wherein the pro-inflammatory cytokine is an Interleukin-type cytokine or a Tumor Necrosis Factor-type cytokine.

4. The method according to claim 3, wherein the Interleukin-type cytokine is selected from the group consisting of Interleukin-Iα (IL-1α), Interleukin-Iβ (IL-Iβ), Interleukin-6 (IL-6), Interleukin-8 (IL-8), Interleukin-11 (IL-II), and Interleukin-18 (IL-18).

5. The method according to claim 3, wherein the Tumor Necrosis Factor-type cytokine is Tumor Necrosis Factor-alpha.

6. The method according to claim 2, wherein the anti-inflammatory cytokine is an Interleukin-type cytokine, a Tumor Necrosis Factor-type cytokine, an Interferon cytokine or a Growth Factor cytokine.

7. The method according to claim 6, wherein the Interleukin-type cytokine is selected from the group consisting of Interleukin-4 (IL-4), Interleukin-10 (IL-10) and Interleukin-13 (IL-13).

8. The method according to claim 6, wherein the Interferon cytokine is IFNα.

9. The method according to claim 6, wherein the Growth Factor cytokine is a Transforming Growth Factor cytokine or a Granulocyte-colony Stimulating Factor cytokine.

10. The method according to claim 6, wherein the Transforming Growth Factor is TGFβ.

11. The method according to claim 6, wherein the Granulocyte-colony Stimulating Factor cytokine is G-CSF.

12. The method according to claim 1, wherein the phosphate derivative of a hydroxy chroman is selected from the group consisting of phosphate derivatives of alpha, beta, delta and gamma tocols in enantiomeric and racemic forms.

13. The method according to claim 12, wherein the tocol phosphate derivative is selected from the group consisting of a tocopheryl phosphate derivative, a tocotrienol phosphate derivative, and a mixture thereof.

14. The method according to claim 13, wherein the tocol phosphate derivative is selected from the group consisting of mono-tocopheryl phosphate derivatives, di-tocopheryl phosphate derivatives, mono-tocotrienyl phosphate derivatives, di-tocotrienyl phosphate derivatives, and mixtures thereof.

15. The method according to claim 14, wherein the tocol phosphate derivative is a mixture of mono-tocopheryl phosphate derivatives, di-tocopheryl phosphate derivatives, mono-tocotrienyl phosphate derivatives, and/or di-tocotrienyl phosphate derivatives.

16. The method according to claim 15, wherein the tocol phosphate derivative is a mixture of mono-tocopheryl phosphate derivatives and di-tocopheryl phosphate derivatives.

17. The method according to claim 16, wherein the tocol phosphate derivative is a mixture of mono-tocopheryl phosphate (TP) and di-tocopheryl phosphate (T2P).

18. The method according to claim 17, wherein the ratio of mono-tocopheryl phosphate (TP) to di-tocopheryl phosphate (T2P) is 4:1 to 1:4, or 2:1 to 1:2, or 2:1.

19. The method according to claim 1, wherein the complex of a phosphate derivative of a hydroxy chroman is formed by a reaction between a phosphate derivative of a hydroxy chroman and a complexing agent.

20. The method according to claim 19, wherein the complexing agent is selected from the group consisting of amphoteric surfactants, cationic surfactants, amino acids having nitrogen functional groups, and proteins containing amino acids having nitrogen functional groups, and proteins selected from the group consisting of insulin, parathyroid hormone (PTH), glucagon, calcitonin, adrenocorticotropic hormone (ACTH), prolactin, Interferon-α and -β and -γ, leutenising hormone (LH), follicle stimulating hormone (FSH), colony stimulating factor (CSF), and growth hormone (GH).

21. The method according to claim 20, wherein the proteins containing amino acids having nitrogen functional groups are proteins having either at least 1 in 62 amino acids as arginine, or at least 1 in 83 histidine, or at least 1 in 65 as lysine, or a form of casein.

22. The method according to claim 20, wherein the complexing agent is a tertiary substituted amine of the formula:
NR⁷R⁸R⁹
wherein R⁷ is selected from the group consisting of C₁₋₂₂ alkyl optionally interrupted by carbonyl; and R⁸ and R⁹ are independently selected from the group consisting of H, CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X in which X is H, Na, K or alkanolamine, provided R⁸ and R⁹ are not both H and when R⁷ is RCO, then R⁸ is CH₃ and R⁹ is (CH₂CH₂)N(C₂H₄OH)—H₂CHOPO₃ or R⁸ and R⁹ together is N(CH₂)₂N(C₂H₄OH) CH₂COO—.

23. The method according to claim 20, wherein the complexing agent is arginine, lysine, or lauryliminodipropionic acid.

24. The method according to claim 1, in which the phosphate derivative of a hydroxy chroman is in the form of a pharmaceutical composition comprising one or more phosphate derivatives of one or more hydroxy chromans and a pharmaceutically acceptable carrier.

25. The method according to claim 24, wherein the pharmaceutical composition is administered by an oral, parenteral, enteral, rectal, vaginal, nasal, inhalation, topical, or ocular administration route.

26.-27. (canceled)

28. A method of modulating inhibiting an inflammatory response and/or stimulating an anti-inflammatory response comprising administering to a subject a therapeutically effective amount of one or more phosphate derivatives of one or more hydroxy chromans, or complexes thereof.

29.-30. (canceled)

31. A method of treatment and/or prophylaxis of immune disorders, inflammatory disorders, and/or cellular proliferative disorders comprising administering to a subject a therapeutically effective amount of one or more phosphate derivatives of one or more hydroxy chromans, or complexes thereof.

32.-33. (canceled)

34. An immune-modulator agent, anti-inflammatory agent, or anti-cancer agent, comprising one or more phosphate derivatives of one or more hydroxy chromans, or complexes thereof.

35.-36. (canceled)
